(19) United States
(12) Patent Application Publication
(10) Pub. No.: US 2026/0240591 A1
(43) Pub. Date: Aug. 20, 2026

(54) MEDICAL DEVICE WITH PUNCTURE ELECTRODE

(71) Applicant: BOSTON SCIENTIFIC SCIMED, INC., Maple Grove, MN (US)

(72) Inventors: Lauren Koon, Etobicoke (CA); Phil Litecky, Forest Lake, MN (US); Eric Michael Petersen, Maple Grove, MN (US)

(73) Assignee: BOSTON SCIENTIFIC SCIMED, INC., Maple Grove, MN (US)

(21) Appl. No.: 19/545,538

(22) Filed: Feb. 20, 2026

Related U.S. Application Data
(60) Provisional application No. 63/760,915, filed on Feb. 20, 2025.

(51) Int. Cl. A61B 18/14; A61B 18/00

(52) U.S. Cl. CPC A61B 18/1477; A61B 2018/00077; A61B 2018/00083; A61B 2018/00178; A61B 2018/00601; A61B 2018/1467

(57) ABSTRACT

A medical device for forming tissue punctures includes an elongate shaft with a distal region and an electrode structure secured to the distal region. The electrode structure contains one or more electrode pairs with first and second electrode segments extending radially from a center point. An electrical conductor extends through the shaft and couples to the electrode structure. The device includes an insulative layer over portions of the electrode structure, with electrode pairs extending above the insulative layer surface to form cutting edges. The electrode structure creates three-dimensional cut patterns generating tissue flaps that fold away from the puncture. The cutting edges may be arranged in various configurations, including spiral patterns, bisecting patterns through the center point, or multiple edges that do not intersect the center point. The device forms cuts sized to accommodate catheter insertion through the resulting puncture.

### CROSS REFERENCE TO RELATED APPLICATIONS

This application claims the benefit of priority of U.S. Provisional Application No. 63/760,915 filed February 20, 2025, the entire disclosure of which is hereby incorporated by reference.

### TECHNICAL FIELD

The disclosure pertains to medical devices and more particularly to medical devices that are adapted to puncture tissue.

### BACKGROUND

A wide variety of medical devices have been developed for medical use. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a medical device that is adapted to form a puncture in tissue. The medical device includes an elongate shaft that extends proximally from a distal region and an electrode structure that is secured to the distal region. The electrode structure includes one or more electrode pairs. Each of the one or more electrode pairs include a first electrode segment and a second electrode segment that each extend radially outwardly from a center point of the electrode structure. An electrical conductor extends through the elongate shaft and is electrically coupled to the electrode structure.

Alternatively or additionally, the medical device may further include an insulative layer disposed over at least a portion of the electrode structure.

Alternatively or additionally, each of the electrode pairs may extend above an outer surface of the insulative layer.

Alternatively or additionally, the electrode structure may taper to a minimum diameter at the center point.

Alternatively or additionally, for each of the one or more electrode pairs the first electrode segment may extend from the center point in a first radial direction and the second electrode segment may extend from the center point in a second radial direction that is 180 degrees apart from the first radial direction.

Alternatively or additionally, for each of the one or more electrode pairs the first electrode segment and the second electrode segment may each extend proximally from the center point.

Alternatively or additionally, the one or more electrode pairs may include a plurality of electrode pairs.

Alternatively or additionally, each of the plurality of electrode pairs may be equally circumferentially arranged about the electrode structure.

Alternatively or additionally, the one or more electrode pairs may include a first electrode pair and a second electrode pair that is orthogonal with the first electrode pair.

Alternatively or additionally, the one or more electrode pairs may include three or more electrode pairs that are equally circumferentially arranged about the electrode structure.

Alternatively or additionally, the one or more electrode pairs may include a primary electrode pair and two or more secondary electrode pairs, where each of the two or more secondary electrode pairs may be parallel with others of the two or more secondary electrode pairs, and they may be orthogonal to the primary electrode pair.

Another example may be found in a medical device that is adapted to form a puncture in tissue. The medical device includes an elongate shaft that extends from a distal region and an electrode structure that is secured to the distal region. The electrode structure includes a center point and is adapted to create a three dimensional cut pattern in tissue creating the puncture. The three dimensional cut pattern creates one or more tissue flaps that are adapted to fold away from the puncture. An electrical conductor extends through the elongate shaft and is electrically coupled to the electrode structure.

Alternatively or additionally, the medical device may further include an insulative layer disposed over at least a portion of the electrode structure.

Alternatively or additionally, the electrode structure may define a cutting edge that extends above an outer surface of the insulating layer.

Alternatively or additionally, the cutting edge may include a spirally arranged cutting edge.

Alternatively or additionally, the cutting edge may bisect the center point of the electrode structure.

Alternatively or additionally, the cutting edge may include a plurality of cutting edges that each bisect the center point of the electrode structure.

Alternatively or additionally, the cutting edge may include a plurality of cutting edges, including one or more cutting edges that do not bisect the center point of the electrode structure.

Another example may be found in a medical device that is adapted to form a puncture in tissue. The medical device includes an elongate shaft that extends proximally from a distal region and an electrode structure that is secured to the distal region. The electrode structure is adapted to form one or more cuts in tissue that together form a puncture sized to accommodate insertion of a catheter through the puncture. An insulative layer extends over at least part of the electrode structure and is adapted to allow portions of the electrode structure to extend above the insulative layer to form cutting edges. An electrical conductor extends through the elongate shaft and is electrically coupled to the electrode structure.

Alternatively or additionally, the cutting edges may include a plurality of paired electrode segments.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a schematic view of a human heart;

FIG. 2 is a schematic view of an illustrative medical device;

FIG. 3 is a schematic end view of an illustrative medical device along with a corresponding cut pattern resulting from use of the illustrative medical device;

FIG. 4 is a schematic end view of an illustrative medical device along with a corresponding cut pattern resulting from use of the illustrative medical device;

FIG. 5 is a schematic end view of an illustrative medical device along with a corresponding cut pattern resulting from use of the illustrative medical device;

FIG. 6 is a schematic end view of an illustrative medical device along with a corresponding cut pattern resulting from use of the illustrative medical device;

FIG. 7 is a schematic end view of an illustrative medical device along with a corresponding cut pattern resulting from use of the illustrative medical device;

FIG. 8 is a schematic view of a distal region of an illustrative medical device shown with its distal region in a biased configuration;

FIG. 9 is a schematic view of the distal region of the illustrative medical device of FIG. 8, shown with the distal region in a configuration in which the distal region has been deflected from the biased configuration; and

FIG. 10 is a perspective view of an illustrative electrode structure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

There are a variety of medical procedures that may include, or otherwise benefit from, being able to form an opening in tissue so that a catheter, guidewire or other medical device may be advanced through the opening. In some cases, there are benefits to forming punctures in valve leaflet tissue such as aortic valve leaflets or mitral valve leaflets. As another example, there are many procedures that include gaining access to the left side of the heart from within the right side of the heart. In some cases, the left atrium may be reached from the right atrium by forming an opening within the atrial septum. In some cases, the left ventricle may be reached from the right ventricle by forming an opening within the ventricular septum. When forming an opening, even temporarily, within the atrial septum and/or the ventricular septum, it can be beneficial to do so in a way that does not cause tissue to be removed from the septum because any debris that reaches the left side of the heart can cause problems for the patient if the debris reaches the brain.

In some cases, a medical device is adapted to form a puncture in tissue. The medical device includes an elongate shaft that extends proximally from a distal region and an electrode structure that is secured to the distal region. The electrode structure includes one or more electrode pairs. Each of the one or more electrode pairs include a first electrode segment and a second electrode segment that each extend radially outwardly from a center point of the electrode structure. An electrical conductor extends through the elongate shaft and is electrically coupled to the electrode structure.

In some cases, the medical device may further include an insulative layer that is disposed over at least a portion of the electrode structure. Each of the electrode pairs may extend above an outer surface of the insulative layer. In some cases, the electrode structure may taper to a minimum diameter at the center point. For each of the one or more electrode pairs, the first electrode segment may extend from the center point in a first radial direction and the second electrode segment may extend from the center point in a second radial direction that is 180 degrees apart from the first radial direction. For each of the one or more electrode pairs, the first electrode segment and the second electrode segment may each extend proximally from the center point.

In some cases, the one or more electrode pairs may include a plurality of electrode pairs. In some cases, each of the plurality of electrode pairs may be equally circumferentially arranged about the electrode structure. In some cases, the one or more electrode pairs may include a first electrode pair and a second electrode pair that is orthogonal with the first electrode pair. In some cases, the one or more electrode pairs may include three or more electrode pairs that are equally circumferentially arranged about the electrode structure. In some cases, the one or more electrode pairs may include a primary electrode pair and two or more secondary electrode pairs. Each of the two or more secondary electrode pairs may be parallel with others of the two or more secondary electrode pairs, and they may be orthogonal to the primary electrode pair.

In some instances, a medical device may be adapted to form a puncture in tissue. The medical device includes an elongate shaft that extends proximally from a distal region and an electrode structure that is secured to the distal region. The electrode structure includes a center point. The electrode structure is adapted to create a three dimensional cut pattern in tissue creating the puncture, where the three dimensional cut pattern creates one or more tissue flaps that are adapted to fold away from the puncture. An electrical conductor extends through the elongate shaft and is electrically coupled to the electrode structure.

In some cases, the medical device may further include an insulative layer that is disposed over at least a portion of the electrode structure. In some case, the electrode structure may define a cutting edge that extends above an outer surface of the insulating layer. In some cases, the cutting edge may include a spirally arranged cutting edge. In some cases, the cutting edge may bisect the center point of the electrode structure. In some cases, the cutting edge may include a plurality of cutting edges that each bisect the center point of the electrode structure. In some cases, the cutting edge may include a plurality of cutting edges, including one or more cutting edges that do not bisect the center point of the electrode structure.

In some instances, a medical device may be adapted to form a puncture in tissue. The medical device includes an elongate shaft that extends proximally from a distal region and an electrode structure that is secured to the distal region. The electrode structure is adapted to form one or more cuts in tissue that together form a puncture sized to accommodate insertion of a catheter through the puncture. An insulative layer extends over at least part of the electrode structure. The insulative layer is adapted to allow portions of the electrode structure to extend above the insulative layer to form cutting edges. An electrical conductor extends through the elongate shaft and is electrically coupled to the electrode structure. In some cases, the cutting edges may include a plurality of paired electrode segments.

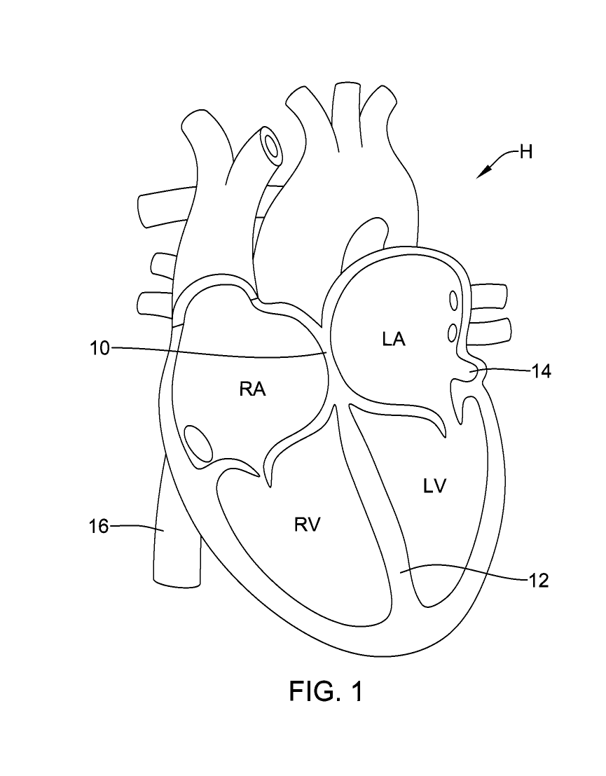

FIG. 1 is a schematic view of a human heart H. The primary chambers of the heart H include a right atrium RA, a right ventricle RV, a left atrium LA and a left ventricle LV. The right atrium RA and the left atrium LA are separated by an atrial septum 10 while the right ventricle RV and the left ventricle LV are separated by a ventricular septum 12. In some instances, the atrial septum 10 and the ventricular septum 12 are together generally referred to as an atrioventricular septum. The left atrium LA includes a left atrial appendage (LAA) 14. An inferior vena cava 16 is fluidly coupled with the right atrium RA. In some cases, the medical devices described herein may be used to form a puncture within the atrial septum 10 and/or the ventricular septum 12.

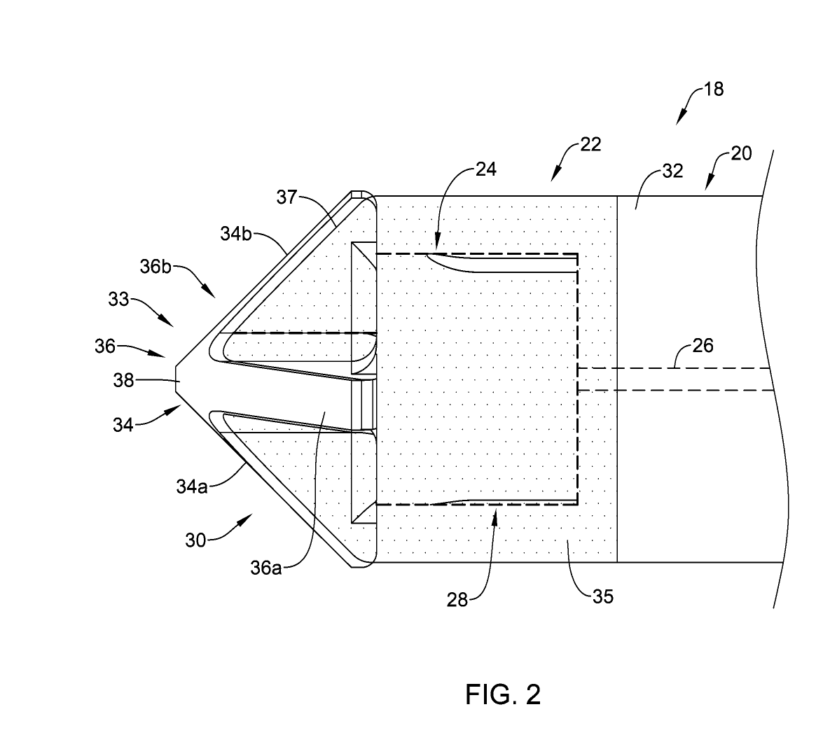

FIG. 2 is a schematic view of an illustrative medical device 18 that may be adapted to puncture tissue. The medical device 18 may be adapted to form a hole or a puncture in a native valve leaflet or in an artificial or replacement valve leaflet such as an aortic valve leaflet or a mitral valve leaflet. In some cases, the medical device 18 may be adapted to form a hole in the atrial septum 10 and/or the ventricular septum 12. The medical device 18 includes an elongate shaft 20 that extends proximally from a distal region 22. An electrode structure 24 is secured to the distal region 22. The electrode 24 may be formed of any biocompatible electrically conductive metal such as platinum, gold, stainless steel, titanium, iridium oxide, or various platinum-iridium alloys. The electrode structure 24 is electrically coupled with an electrical conductor 26 that extends through the elongate shaft 20. In some cases, the electrical conductor 26 may extend proximally to a handle (not shown) that includes a feature for connecting a source of RF (radiofrequency) energy to the electrical conductor 26, and hence to the electrode structure 24.

The electrode structure 24 includes a proximal portion 28 and a distal portion 30. In some cases, the proximal portion 28 may be embedded within a sheath 32 that forms part of the elongate shaft 20. In some cases, the proximal portion 28 may be covered by an electrically insulating layer 35. The electrically insulating layer 35 may extend over part of the distal portion 30 as well. The electrically insulating layer 35 may be formed of any of a variety of suitable polymers. Examples of suitable polymers for forming the electrically insulating layer 35 include PTFE (polytetrafluoroethylene), commonly known as Teflon, ETFE (ethylene tetrafluoroethylene), polyurethane, silicone rubber, polyimide, and polyethylene. As shown, distal portion 30 of the electrode structure 24 may be considered as including cutting edges 33 that may be used to cut through tissue via applied RF energy. In some cases, the cutting edges 33 extend above an outer surface 37 of the electrically insulating layer 35. In some cases, the cutting edges 33 may be considered as including one or more electrode pairs. As shown, there is an electrode pair 34 that includes a first electrode segment 34a and a second electrode segment 34b and an electrode pair 36 that includes a first electrode segment 36a and a second electrode segment 36b (hidden from view on back side of the electrode structure 24). In some cases, each of the electrode segments 34a, 34b, 36a, and 36b each extend radially outwardly from a center point 38. In some cases, the electrode pair 34 and the electrode pair 36 each bisect the center point 38. In some cases, the electrode structure 24 may be considered as being pyramidal in shape, with straight edges and lines. In some cases, the electrode structure 24 may include rounded edges. In some cases, the electrically insulating layer 35 may be round or curved, for example. In some cases, having the electrode structure include rounded edges and shapes can make the electrode structure 24 more atraumatic and can reduce instances of auto-puncture resulting from catheter pressure.

In some cases, as shown, the electrode structure 24 tapers to a minimum diameter at the center point 38. In some cases, for each of the one or more electrode pairs (such as the electrode pair 34 and the electrode pair 36), one of the electrode segments extends from the center point 38 in a first radial direction and the other of the electrode segments extend from the center point 38 in a second radial direction that is 180 degrees apart from the first radial direction. This may be seen, for example, in the first electrode segment 34a, which extends from the center point 38 in a downward (in the illustrated orientation) direction and in the second electrode segment 34b, which extends from the center point 38 in an upward (in the illustrated orientation) direction. Because the electrode structure 24 is tapered, each of the first electrode segment 34a and the second electrode segment 34b (and the first electrode segment 36a and the unseen second electrode segment 36b) extend both radially outwardly but also in a proximal direction.

FIG. 2 shows an electrode structure 24 that includes two electrode pairs 34 and 36. In some cases, the medical device 18 may include an electrode structure including one electrode pair or three or more electrode pairs. In some cases, each of the plurality of electrode pairs may be equally circumferentially spaced about the electrode structure. In some cases, the medical device 18 may include an electrode structure including other arrangements of electrode pairs and/or cutting edges 33. In some cases, an electrode structure may not have matched pairs of electrode pairs, but may instead include an odd number of electrode segments. As an example, an electrode structure may include 3+2n electrode segments, where n is an integer equal to or greater than one. In some cases, an odd number of electrode segments may be arranged in a radially symmetric manner. For example, a total of five electrode segments (with n=1) may be arranged such that each of the five electrode segments are circumferentially spaced about 72 degrees. In some cases, an electrode structure may include 1+2n electrode segments, where n is an integer equal to or greater than one.

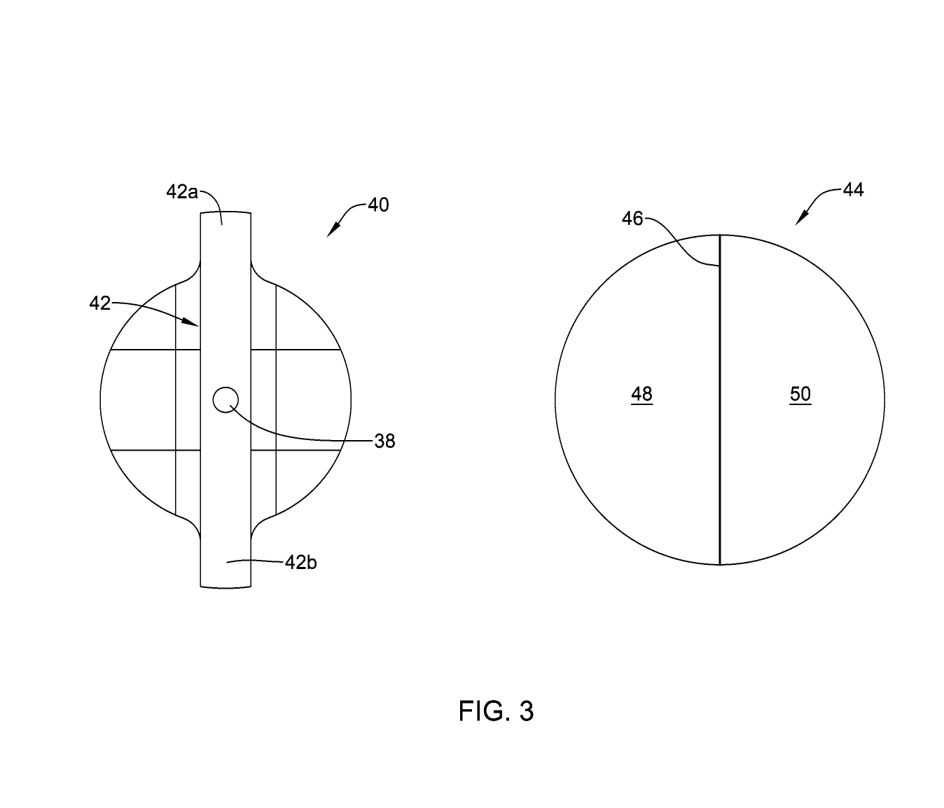

The left side of FIG. 3 shows an illustrative electrode structure 40 including a single electrode pair 42. The electrode structure 40 may be used as part of the medical device 18, in place of the electrode structure 24, for example. The electrode pair 42 includes a first electrode segment 42a and a second electrode segment 42b. The right side of FIG. 3 is a schematic view 44 of a corresponding cutting pattern created using the electrode structure 40 when the electrode structure 40 is urged against tissue and energized with RF energy. The single electrode pair 42 has resulted in a single cut 46. In some cases, the single cut 46 may have a length that is at least as great as a diameter of a catheter or other device to subsequently be inserted through the single cut 46. The tissue on either side of the single cut 46 may be considered as forming a flap 48 and a flap 50. The flap 48 and the flap 50 are not removed, but remain on either side of the single cut 46 and may close back together subsequent to a catheter or other device being inserted through the single cut 46.

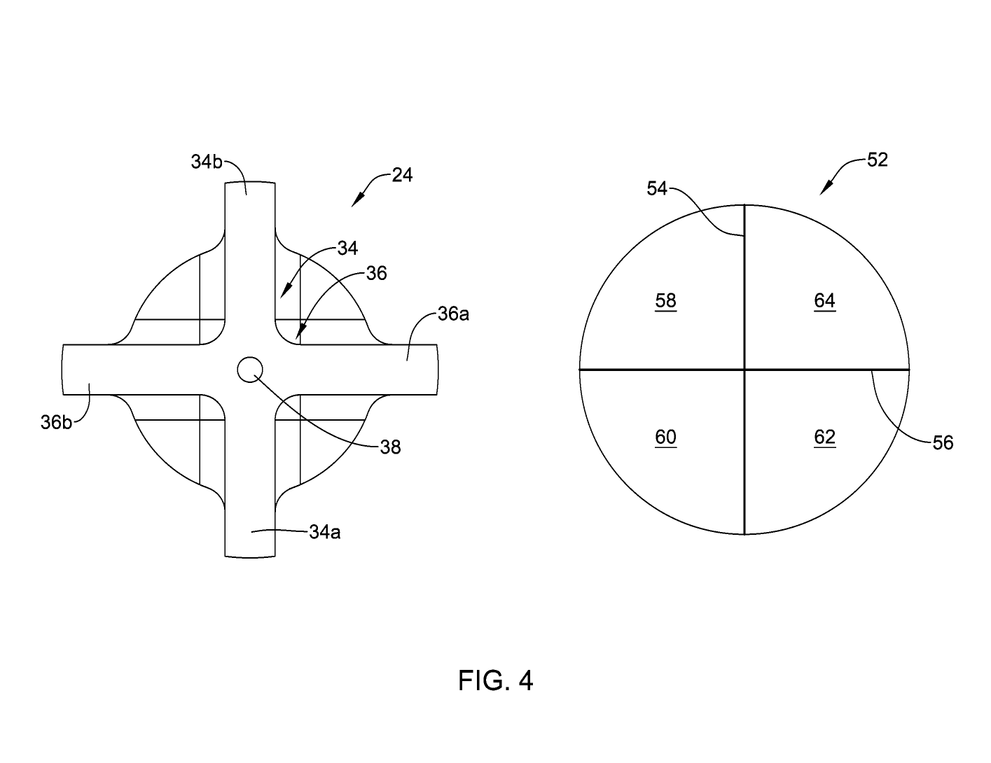

The left side of FIG. 4 may be considered as being an end view of the medical device 18 shown in FIG. 2. As shown, the electrode structure 24 includes the electrode pair 34, including the first electrode segment 34a and the second electrode segment 34b, and the electrode pair 36, including the first electrode segment 36a and the second electrode segment 36b. The right side of FIG. 4 is a schematic view of a corresponding cut pattern 52 formed by the electrode structure 24 when the electrode structure 24 is urged against tissue and energized with RF energy. The first electrode pair 34 may be seen as having formed the cut 54 and the second electrode pair 36 may be seen as having formed the cut 56. The cuts 54 and 56 divide the tissue into four flaps 58, 60, 62, and 64. The flaps 58, 60, 62, and 64 are not removed, but remain in place when not pushed out of the way by a catheter or other device being extended through the cuts 54 and 56.

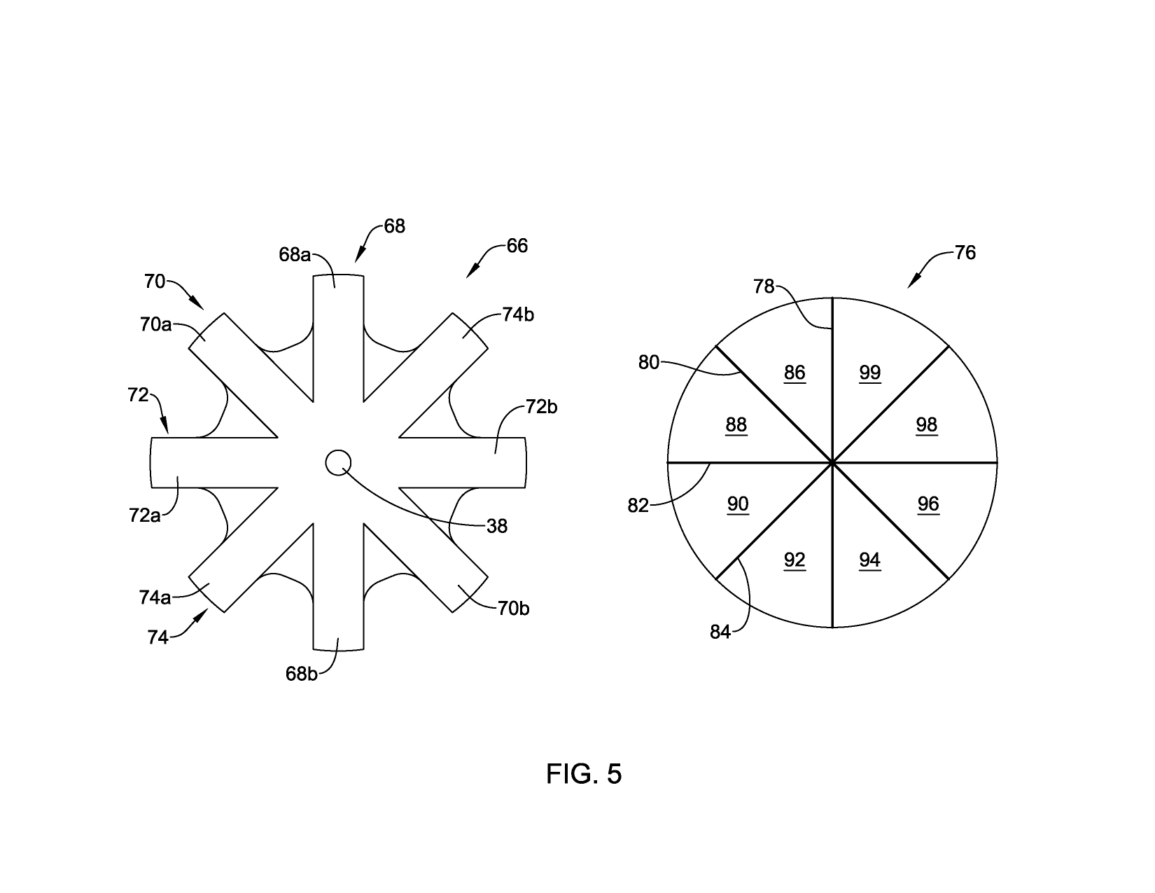

The left side of FIG. 5 shows an electrode structure 66 that may be used as part of the medical device 18, in place of the electrode structure 24, for example. The electrode structure 66 includes a first electrode pair 68 having a first electrode segment 68a and a second electrode segment 68b, a second electrode pair 70 having a first electrode segment 70a and a second electrode segment 70b, a third electrode pair 72 having a first electrode segment 72a and a second electrode segment 72b, and a fourth electrode pair 74 having a first electrode segment 74a and a second electrode segment 74b. The right side of FIG. 5 is a schematic view of a corresponding cut pattern 76 formed by the electrode structure 66 when the electrode structure 66 is urged against tissue and energized with RF energy. The first electrode pair 68 may be seen as having formed a cut 78, the second electrode pair 70 may be seen as having formed a cut 80, the third electrode pair 72 may be seen as having formed a cut 82, and the fourth electrode pair 74 may be seen as having formed a cut 84. The cuts 78, 80, 82, and 84 divide the tissue into flaps 86, 88, 90, 92, 94, 96, 98, and 99. The flaps 86, 88, 90, 92, 94, 96, 98, and 99 are not removed, but remain in place when not pushed out of the way by a catheter or other device being extended through the cuts 78, 80, 82, and 84.

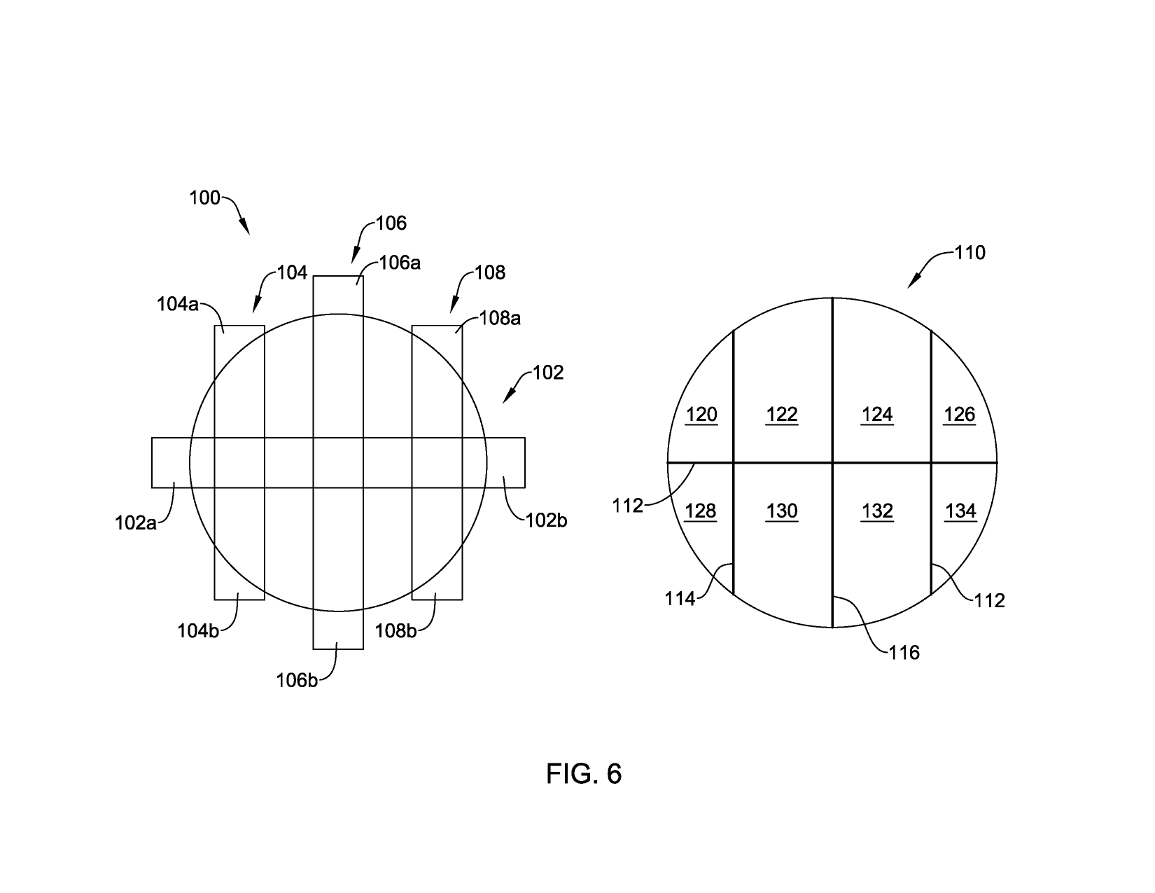

The left side of FIG. 6 shows an electrode structure 100 that may be used as part of the medical device 18, in place of the electrode structure 24, for example. The electrode structure 100 includes a primary electrode pair 102 having a first electrode segment 102a and a second electrode segment 102b. The electrode structure 100 includes two or more secondary electrode pairs including a first secondary electrode pair 104 having a first electrode segment 104a and a second electrode segment 104b, a second secondary electrode pair 106 having a first electrode segment 106a and a second electrode segment 106b, and a third secondary electrode pair 108 having a first electrode segment 108a and a second electrode segment 108b. In some cases, each of the secondary electrode pairs 104, 106, and 108 may be parallel with each other and may each be orthogonal to the primary electrode pair 102.

The right side of FIG. 6 shows a schematic view of a corresponding cut pattern 110 formed by the electrode structure 100 when the electrode structure 100 is urged against tissue and energized with RF energy. The primary electrode pair 102 may be seen as having formed a cut 112, the first secondary electrode pair 104 may be seen as forming a cut 114, the first secondary electrode pair 106 may be seen as forming a cut 116, and the second secondary electrode pair 108 may be seen as forming a cut 118. The cuts 112, 114, 116, and 118 divide the tissue into flaps 120, 122, 124, 126, 128, 130, 132, and 134. The flaps 120, 122, 124, 126, 128, 130, 132, and 134 are not removed, but remain in place when not pushed out of the way by a catheter or other device being extended through the cuts 112, 114, 116, and 118.

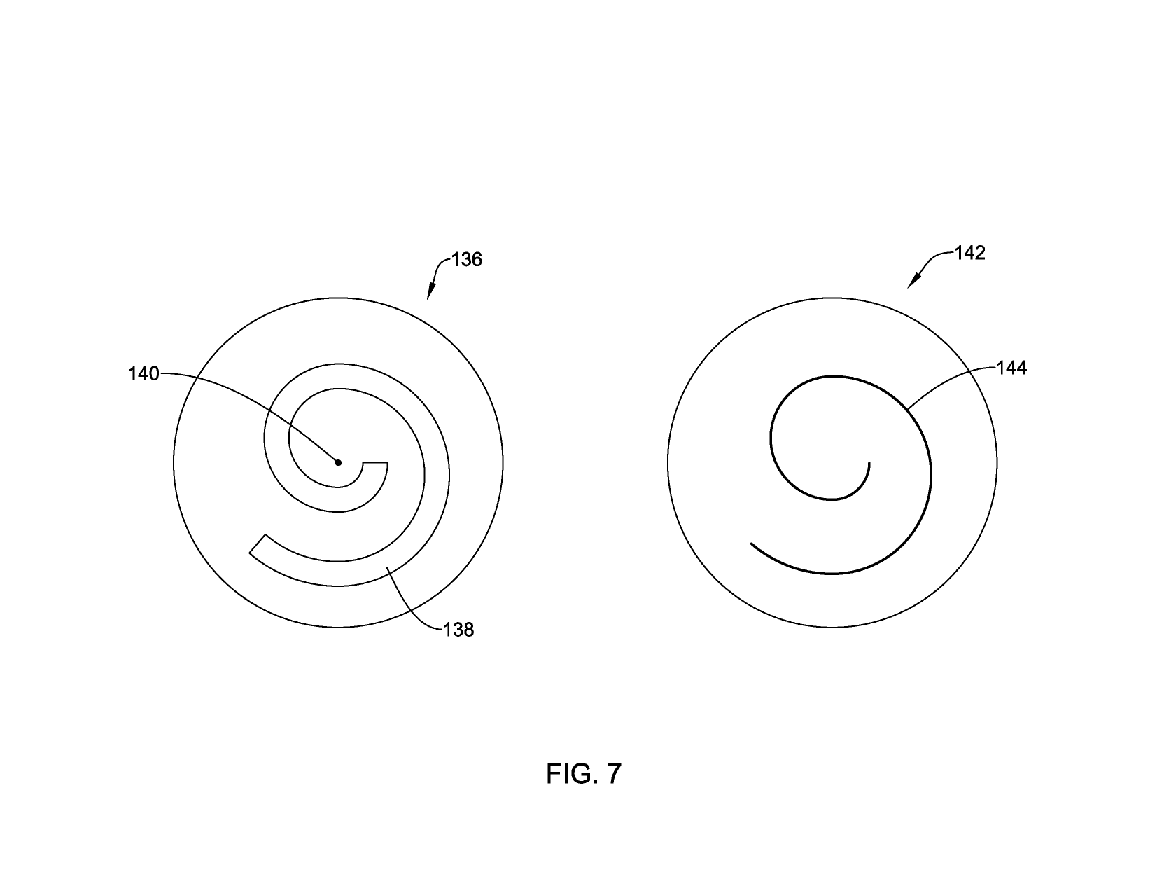

The left side of FIG. 7 shows an electrode structure 136 that may be used as part of the medical device 18, in place of the electrode structure 24, for example. The electrode structure 136 includes a cutting edge 138 that is arranged in a spiral. In some cases, the cutting edge 138 spirals in a circumferential direction. In some cases, the cutting edge 138 also spirals in a longitudinally direction by virtue of the electrode structure 136 tapering down to a minimum diameter at a center point 140. The right side of FIG. 7 shows a schematic view of a cutting pattern 142 formed by the electrode structure 136 when the electrode structure 136 is urged against tissue and energized with RF energy. The cutting pattern 142 includes a cut 144 that extends in a spiral direction.

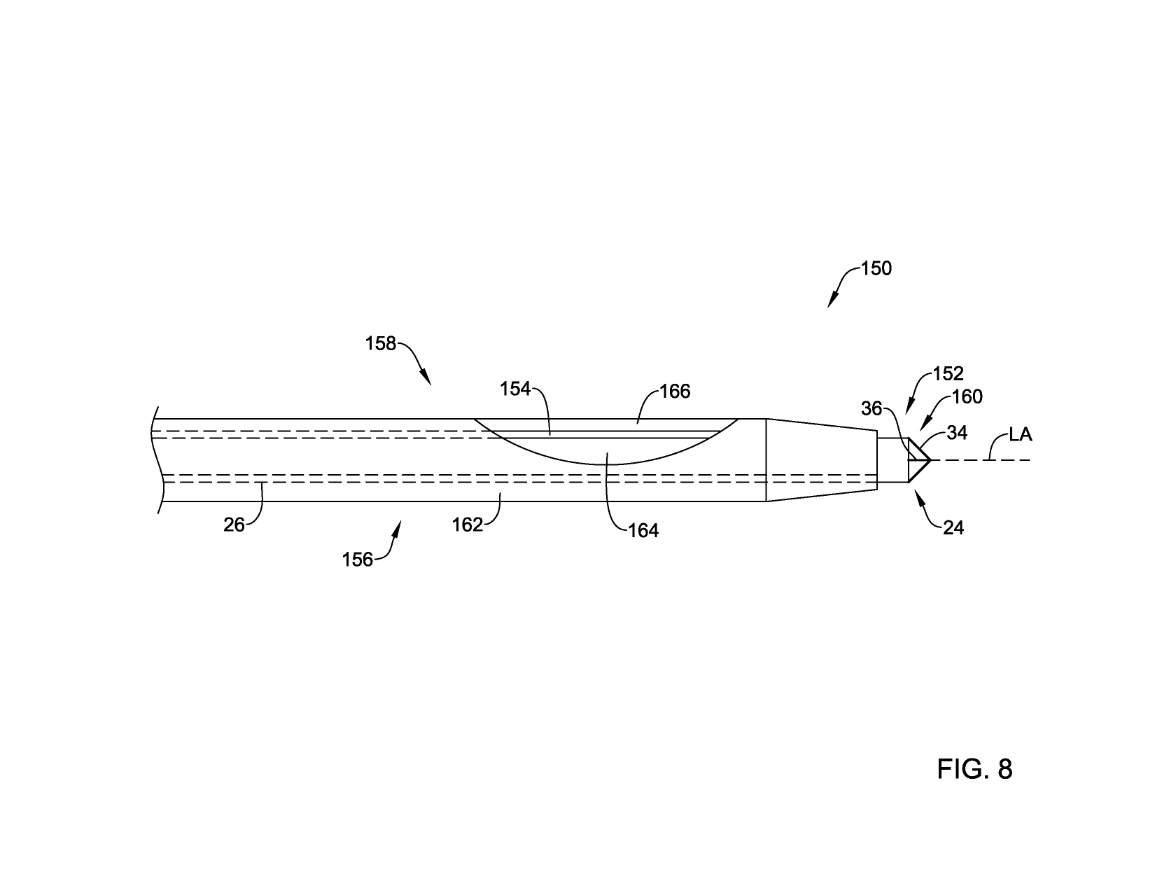

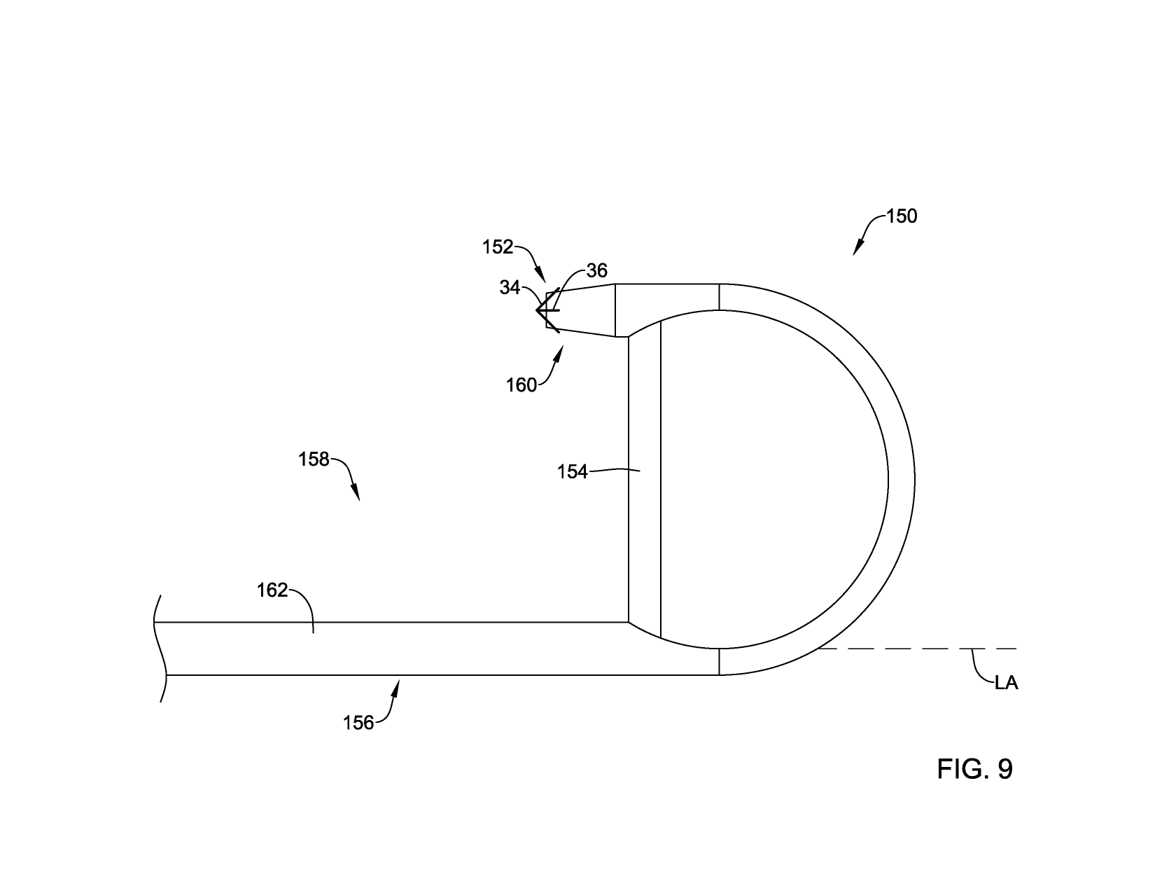

FIGS. 8 and 9 are schematic views of an illustrative medical device 150. The illustrative medical device 150 may be used for lacerating a valve leaflet such as a native valve leaflet or an artificial or replacement valve leaflet. A number of patients receive artificial heart valves for a variety of reasons including valve malfunction due to calcium accumulation. When an artificial heart valve is implanted, the artificial heart valve may have an expandable frame that presses the native valve leaflets away from the native position of the native valve leaflets. In some instances, the native valve is the aortic valve, and the artificial heart valve is an artificial aortic valve. In some instances, it is possible for one or more of the native valve leaflets, when pressed to the side, to at least partially or even completely block an ostium of one of the coronary arteries. Not only does this present possible health concerns for the patient, particularly if an ostium is completely blocked, but even when an ostium is only partially blocked and thus still permits blood flow, this may present difficulties in subsequently being able to perform balloon angioplasty, or place a stent, in one of the coronary arteries. In some instances, it may be beneficial to slice or lacerate with opportunity to remove one or more of the native valve leaflets prior to implantation of the artificial heart valve so that when the native valve leaflets are pressed to the side by the expandable frame of the artificial heart valve, the native valve leaflets do not block an ostium of any of the coronary arteries.

In some instances, a patient may already have an implanted artificial heart valve such as an artificial aortic valve. The artificial valve leaflets forming part of the already implanted artificial heart valve can be just as problematic with respect to potentially blocking a cardiac artery ostium when displaced to the side when a second artificial heart valve is implanted in place of the first artificial heart valve. The artificial valve leaflets forming part of the artificial heart valve may, for example, be made from porcine or bovine tissue, or may be polymeric. In some instances, artificial valve leaflets may be made of polymers such as Dacron or Gore-Tex. As discussed here, reference to a valve leaflet may refer to either a native valve leaflet or an artificial valve leaflet.

As noted, the medical devices described herein may be used in lacerating valve leaflets regardless of whether the valve leaflets are native valve leaflets or artificial valve leaflets. In some cases, the native valve leaflets may be lacerated prior to implantation of an artificial heart valve in order to avoid possible issues with one or more of the native valve leaflets from obscuring an ostium of one of the coronary arteries. Even if blood is able to flow through the ostium and into one of the coronary arteries, having the ostium even partially blocked with a native valve leaflet can potentially cause difficulties with subsequent procedures such as performing angioplasty within one of the coronary arteries or implanting a stent within one of the coronary arteries.

In some cases, a second artificial heart valve may be implanted within a previously implanted artificial heart valve. There may be a desire to lacerate one or more of the artificial valve leaflets within the previously implanted artificial heart valve before implanting the replacement artificial heart valve within the previously implanted artificial heart valve. In some cases, lacerating one or more of the artificial valve leaflets may help reduce or eliminate potential issues with the artificial valve leaflets of the previously implanted artificial heart valve interfering with operation of the replacement artificial heart valve and/or potentially blocking an ostium of one of the coronary arteries.

The medical device 100 may incorporate any of the electrical structures 24, 40, 66, 100, and 136, and variations thereof, as described herein. The medical device 150 may include a piercing electrode 152 and a lacerating electrode 154. In some cases, the piercing electrode 152 may be used to form a puncture or hole within a valve leaflet such as a native valve leaflet or a replacement valve leaflet, followed by advancing the medical device 100 through the puncture so that the lacerating electrode 154 may be used to lacerate the valve leaflet. In some cases, the piercing electrode 152 may incorporate any of the electrical structures 24, 40, 66, 100, and 136, and variations thereof, as described herein, as the piercing electrode 152.

The medical device 150 includes an elongate shaft 156 that extends proximally from a distal region 158. In some cases, a handle (not shown) may be secured relative to the elongate shaft 156. The distal region 158 may be considered as being a deflectable distal region 158. The piercing electrode 152 may be disposed at a distal end 160 of the distal region 158. In some cases, the piercing electrode 152 is stationary relative to the distal end 160. In some cases, the piercing electrode 152 may be axially translatable relative to the distal end 160. As an example, the piercing electrode 152 may be axially advanceable relative to the distal end 160, and can be extended for use and withdrawn into the distal end 160 when not in use.

In some cases, the distal region 158 of the elongate shaft 156 has an outer surface 162 that extends along the elongate shaft 156. As shown, an elongate slot 164 may be formed within the outer surface 162. The elongate slot 164 may extend in a direction parallel or at least substantially parallel (defined as within ten percent) with a longitudinal axis LA and exposes a lumen 166 extending within the distal region 158 of the elongate shaft 156. In some cases, the lacerating electrode 154 extends through the lumen 166 and is at least partially exposed by the elongate slot 164. As shown, the distal region 158 of the elongate shaft 156 may be considered as being in a biased configuration, meaning that absent external forces, this is the configuration that the distal region 158 of the elongate shaft 156 will achieve. In this example, the piercing electrode 152 utilizes the electrode structure 24 and thus includes the first electrode pair 34 and the second electrode pair 36, although any of the electrode structures 40, 66, 100, and 136, and variations thereof, may be used as the piercing electrode 152. The electrical structure 24 is electrically coupled with the electrical conductor 26.

In some cases, the distal region 158 of the elongate shaft 156 may be deflected from the biased configuration (shown in FIG. 8) as part of actuating and then energizing the lacerating electrode 154. FIG. 9 shows the distal region 158 of the elongate shaft 156 deflected from the biased configuration into a configuration in which the lacerating electrode 154 is positioned to lacerate tissue. In some cases, when the distal region 158 of the elongate shaft 156 is deflected away from the biased configuration, the lacerating electrode 154 may form an angle of about 45 degrees to about 135 degrees with the longitudinal axis LA. As shown, the lacerating electrode 154 is forming an angle of about 90 degrees with the longitudinal axis LA. When in this configuration, the medical device 150 may be pulled proximally while the lacerating electrode 154 is energized in order to cut or lacerate tissue or other materials.

While not expressly shown, the lacerating electrode 154 may have a distal end that is secured at or near the distal end 160 of the distal region 158. Pulling on the lacerating electrode 154 exerts a tensile force on the lacerating electrode 154 that will exert a tensile force on the distal end 160 of the distal region 158. In response, the distal region 158 of the elongate shaft 156 will deflect in response to the applied tensile force. In some cases, inclusion of the elongate slot 164 will cause the distal region 158 of the elongate shaft 156 to preferentially bend in the direction indicated in FIG. 9 because the elongate slot 164 removes material from the distal region 158 of the elongate shaft 156. The elongate shaft 156 also permits the portion of the lacerating electrode 154 passing underneath the elongate slot 164 when in the biased configuration to extend out of the elongate slot 164 when deflected from the biased configuration. Additional details regarding the medical device 150, including a possible handle design and electronics selectively energizing the piercing electrode 152 and the lacerating electrode 154, may be found in U.S. Provisional Patent Application Serial Number 63/674,000 filed July 22, 2024 and entitled LEAFLET MODIFICATION DEVICE WITH INDEPENDENTLY ACUATED PIERCING ELECTRODE AND LACERATING ELECTRODE, which application is incorporated by reference in its entirety.

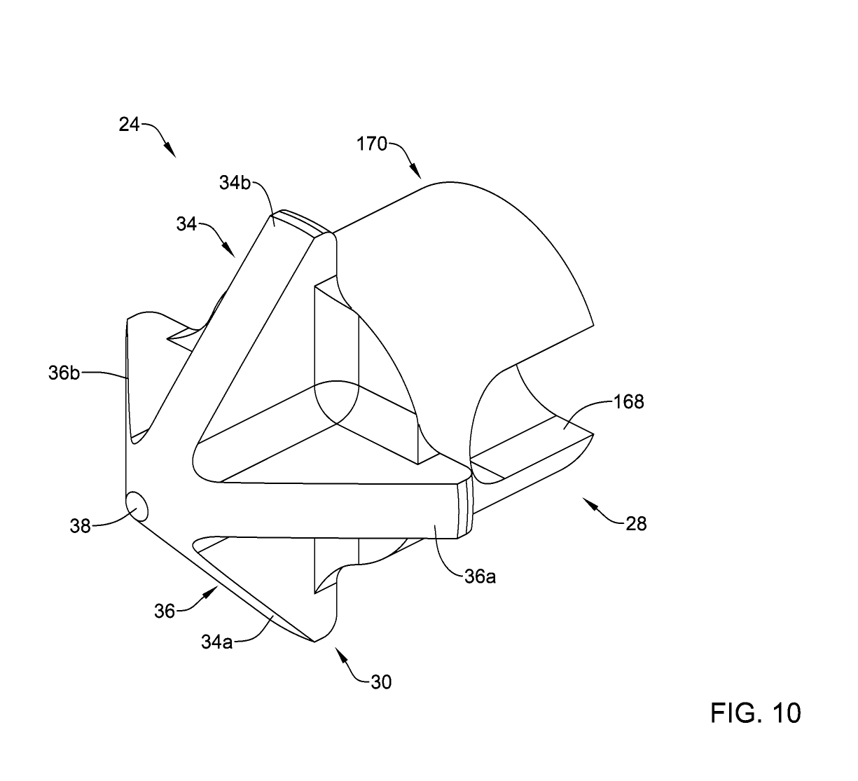

FIG. 10 is a perspective view of the electrode structure 24, shown without the electrically insulative layer 35 in order to provide a better view of the underlying structure forming the electrode pair 34 and the electrode pair 36. One feature of the electrode structure 24 is that the proximal portion 28 is configured to accommodate a distal end of the lacerating electrode 154 and a distal end of the electrical conductor 26. The proximal portion 28 may be seen as including a recess 168 and a recess 170 that is not visible in this view, but is disposed opposite of the recess 168. A firm coupling with the lacerating electrode 154 permits the elongate shaft 156 to deflect when a tensile force is applied to a proximal end of the conductive member forming the lacerating electrode 154. In some cases, a distal portion of the lacerating electrode 154 that engages one of the recesses 168 and 170 may be electrically insulated while a distal portion of the electrical conductor 26 is not electrically insulated so that the electrical conductor 26 is able to make an electrical connection with the electrode structure 24.

The materials that can be used for the various components of the devices described herein may include those commonly associated with medical devices. In some instances, the various components of the devices described herein may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some instances, a degree of Magnetic Resonance Imaging (MRI) compatibility is imparted into the devices and components thereof disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel- cobalt-chromium-molybdenum alloys (e.g., UNS: R30035 such as MP35-N® and the like), nitinol, and the like, and others.

In some instances, the devices and components thereof may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some instances, the devices and components thereof disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni-Co-Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some instances, the devices and components thereof disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A medical device adapted to form a puncture in tissue, the medical device comprising:
an elongate shaft extending proximally from a distal region;
an electrode structure secured to the distal region, the electrode structure comprising:
one or more electrode pairs, each of the one or more electrode pairs including a first electrode segment and a second electrode segment that each extend radially outwardly from a center point of the electrode structure; and
an electrical conductor extending through the elongate shaft and electrically coupled to the electrode structure.

2. The medical device of claim 1, further comprising an insulative layer disposed over at least a portion of the electrode structure.

3. The medical device of claim 2, wherein each of the electrode pairs extend above an outer surface of the insulative layer.

4. The medical device of claim 1, wherein the electrode structure tapers to a minimum diameter at the center point.

5. The medical device of claim 1, wherein for each of the one or more electrode pairs the first electrode segment extends from the center point in a first radial direction and the second electrode segment extends from the center point in a second radial direction that is 180 degrees apart from the first radial direction.

6. The medical device of claim 5, wherein for each of the one or more electrode pairs the first electrode segment and the second electrode segment each extend proximally from the center point.

7. The medical device of claim 1, wherein the one or more electrode pairs comprise a plurality of electrode pairs.

8. The medical device of claim 7, wherein each of the plurality of electrode pairs are equally circumferentially arranged about the electrode structure.

9. The medical device of claim 1, wherein the one or more electrode pairs comprise a first electrode pair and a second electrode pair that is orthogonal with the first electrode pair.

10. The medical device of claim 1, wherein the one or more electrode pairs comprise three or more electrode pairs that are equally circumferentially arranged about the electrode structure.

11. The medical device of claim 1, wherein the one or more electrode pairs comprise a primary electrode pair and two or more secondary electrode pairs, where each of the two or more secondary electrode pairs are parallel with others of the two or more secondary electrode pairs, and are orthogonal to the primary electrode pair.

12. A medical device adapted to form a puncture in tissue, the medical device comprising:
an elongate shaft extending proximally from a distal region;
an electrode structure secured to the distal region, the electrode structure including a center point, the electrode structure adapted to create a three dimensional cut pattern in tissue creating the puncture, the three dimensional cut pattern creating one or more tissue flaps that are adapted to fold away from the puncture; and
an electrical conductor extending through the elongate shaft and electrically coupled to the electrode structure.

13. The medical device of claim 12, further comprising an insulative layer disposed over at least a portion of the electrode structure.

14. The medical device of claim 13, wherein the electrode structure defines a cutting edge that extends above an outer surface of the insulating layer.

15. The medical device of claim 14, wherein the cutting edge comprises a spirally arranged cutting edge.

16. The medical device of claim 14, wherein the cutting edge bisects the center point of the electrode structure.

17. The medical device of claim 14, wherein the cutting edge comprises a plurality of cutting edges that each bisect the center point of the electrode structure.

18. The medical device of claim 14, wherein the cutting edge comprises a plurality of cutting edges, including one or more cutting edges that do not bisect the center point of the electrode structure.

19. A medical device adapted to form a puncture in tissue, the medical device comprising:
an elongate shaft extending proximally from a distal region;
an electrode structure secured to the distal region, the electrode structure adapted to form one or more cuts in tissue that together form a puncture sized to accommodate insertion of a catheter through the puncture;
an insulative layer extending over at least part of the electrode structure, the insulative layer adapted to allow portions of the electrode structure to extend above the insulative layer to form cutting edges; and
an electrical conductor extending through the elongate shaft and electrically coupled to the electrode structure.

20. The medical device of claim 19, wherein the cutting edges comprise a plurality of paired electrode segments.
